(19) United States
(12) Patent Application Publication
(10) Pub. No.: US 2008/0081816 A1
(43) Pub. Date: Apr. 3, 2008

(54) ANTI-INFLAMMATION ACTIVITY OF NEWLY SYNTHESIZED XANTHINE DERIVATIVES KMUP-1 AND KMUP-3

(75) Inventors: Ing-Jun Chen, Kaohsiung City (TW); Bin-Nan Wu, Kaohsiung City (TW); Jwu-Lai Yeh, Kaohsiung City (TW)

Correspondence Address: VOLPE AND KOENIG, P.C., UNITED PLAZA, SUITE 1600, 30 SOUTH 17TH STREET, PHILADELPHIA, PA, 19103, US

(73) Assignee: KAOHSIUNG MEDICAL UNIVERSITY, Kaohsiung City (TW)

(21) Appl. No.: 11/538,236

(22) Filed: Oct. 3, 2006

(51) Int. Cl. A61K 31/522; C07D 473/04

(52) U.S. Cl. 514/252.16; 544/270

(57) ABSTRACT

An anti-inflammation substrate for decreasing the proinflammation induced by the cytokines and inhibiting the lung function degeneration is provided. The anti-inflammation substrate includes one selected from the group consisting of a 7-[2-[4-(2-chlorobenzene)piperazinyl]ethyl]-1,3-dimethylxanthine, a 7-[2-[4-(4-nitrobenzene)piperazinyl]ethyl]-1,3-dimethylxanthine, a respective pharmaceutical acceptable salt thereof, and a combination thereof.

### FIELD OF THE INVENTION

The present invention relates to newly synthesized anti-inflammatory xanthine derivatives KMUP-1 and KMUP-3 for decreasing the proinflammation induced by the cytokines and inhibiting the lung function degeneration.

### BACKGROUND OF THE INVENTION

Pro-inflammatory cytokines, including the tumor necrosis factor-α, TNF-α, play an important role in regulating the tracheal smooth muscle contractility that is found in the asthmatic phenotype. It has been reported that the TNF-α is increased in the sputa of patients with bronchial asthma and present in the broncoalveolar lavage fluid of symptomatic asthmatics. As a member of these cytokines, TNF-α attracting and activating non-specific inflammatory macrophages and neutrophils during infection and hypersensitivity induced by the inhalation of organic particles or fumes has also been reported.

Likewise, the pro-inflammatory TNF-α, the inducible nitric oxide synthase (iNOS) and the cyclooxygenase-2 (COX-2) are co-expressed in pulmonary airway infection. A local production of TNF-α has been found to be regulated by iNOS and COX-2 and thus the level of TNF-α serves to orchestrate the inflammation pathway.

The enhanced COX-2 and iNOS expression by TNF-α can increase the production of cAMP and cGMP due to the activated adenylate cyclase and guanylate cyclase, respectively. Since high-output cyclic nucleotide production in response to inflammation suppresses protein kinase G (PKG) expression, and cAMP analogs are more potent than cGMP analogs in reducing PKG mRNA expression, suggesting that PKA mediated the effects of cAMP and cGMP through cross-activation.

A non-xanthine-based activator, (YC-1), effective in inhibiting the in the lung epidermal cell and increasing the COX-2 expression.

A non-xanthine soluble guanylyl cyclase (sGC) activator, 1-benzyl-1-3-(5′-hydroxymehyl 1-2′-furyl)indazol), YC-1, has been reported exerting cGMP-dependent and cGMP-independent actions, where the cGMP-independent actions include the inhibition of phosphodiesterase (PDE) and untoward COX-2 expression in pulmonary epithelial cells. PDE5 inhibitors with cGMP-increasing activity have proven to induce the tracheal relaxation. One of them, sildenafil was found to induce eNOS and delay preconditioning through iNOS-dependent pathway.

However, the pro-inflammatory iNOS and COX-2 is undesirable when researching new and safe tracheal relaxants. Hence, the YC-1 and the sildenafil are not desirable for serving as safe tracheal relaxants in view of the inflammatory defects thereof.

Based on the above, to develop more potent sGC activators or cGMP level enhancers, which are free from the increased expression of the pro-inflammatory iNOS and COX-2 respectively persisting in the YC-1 and sildenafil, has become a major subject in this art.

In order to overcome the drawbacks in the prior art, the novel xanthine-based sGC activators or cGMP level enhancers with the tracheal relaxation and anti-inflammation properties are provided. The particular design in the present invention not only solves the problems described above, but also is easy to be implemented. Thus, the invention has the utility for the industry.

### SUMMARY OF THE INVENTION

The present invention provides the possible mechanisms of the xanthine-based 7-[2-[4-(2-chlorobenzene)piperazinyl]ethyl]-1,3-dimethylxanthine and 7-[2-[4-(4-nitrobenzene)piperazinyl]ethyl]-1,3-dimethylxanthine, where the two mentioned xanthine-based compounds inhibit TNF-α-induced expression of iNOS in tracheal smooth muscle cells (TSMCs), involving sGC/cGMP/PKG expression pathway, but without the involvement of COX-2.

In accordance with one aspect of the present invention, an anti-inflammation substrate is provided. The anti-inflammation substrate includes one selected from the group consisting of a 7-[2-[4-(2-chlorobenzene)piperazinyl]ethyl]-1,3-dimethylxanthine, a 7-[2-[4-(4-nitrobenzene)piperazinyl]ethyl]-1,3-dimethylxanthine, a respective pharmaceutical acceptable salt thereof, and a combination thereof.

Preferably, the anti-inflammation substrate further has one of an epithelium-derived nitric oxide enhancing activity and an endothelium-derived nitric oxide enhancing activity.

Preferably, the anti-inflammation substrate further imcludes one selected from the group consisting of a pharmaceutical excipient, a diluent and a carrier.

Preferably, the anti-inflammation substrate is further used for effecting in a tracheal cGMP accumulation and relaxing a tracheal constriction by an activation of a soluble guanylate cyclase and an inhibition of the phosphodiesterase.

Preferably, the anti-inflammation substrate is further used for preventing an airway constriction induced by a tissue necrosis factor-α by an activation of a soluable guanylate cyclase, increasing a release of cGMP and activating a protein kinase G.

Preferably, the anti-inflammation substrate is further used for reversing a proinflammation induced by a tissue necrosis factor-α and inhibiting a lung function degeneration.

Preferably, the anti-inflammation substrate is further used for inhibiting an inducible nitric oxide synthase (iNOS) and a protein kinase A activities and a NO production in a lung.

Preferably, the anti-inflammation substrate is further used for preventing a soluable guanylate cyclase and a protein kinase G expression from decreasing.

Preferably, the anti-inflammation substrate is a xanthine-based anti-proinflammation substrate.

Preferably, the anti-inflammation substrate further inhibits the inflammation in one selected from a group consisting of a respiratory airway, a trachea and a blood vessel in a human body, wherein the inflammation comprising a pro-inflammation.

In accordance with the other aspect of the present invention, a method for inhibition a proinflammation induced by a tissue necrosis factor-α in a mammal tracheal smooth muscle cell is provided. The method includes administering to the mammal tracheal smooth muscle cell an inhibition-effective amount of a substrate selected from a group consisting of a 7-[2-[4-(2-chlorobenzene)piperazinyl]ethyl]-1,3-dimethylxanthine, 7-[2-[4-(4-nitrobenzene)piperazinyl]ethyl]-1,3-dimethylxanthine, a respective pharmaceutical acceptable salt thereof, and a combination thereof.

In accordance with the other aspect of the present invention, an anti-inflammatory use in treating one of a chronic obstructive pulmonary disease (COPD) and an asthma is provided, where the use includes administering an pharmaceutically effective amount of a substrate selected from a group consisting of a 7-[2-[4-(2-chlorobenzene)piperazinyl]ethyl]-1,3-dimethylxanthine, 7-[2-[4-(4-nitrobenzene)piperazinyl]ethyl]-1,3-dimethylxanthine, a respective pharmaceutical acceptable salt thereof, and a combination thereof.

In accordance with the further aspect of the present invention, a method for synthesizing an anti-inflammation substrate is provided. The method inludes providing a compound selected from the group consisting of a 7-[2-[4-(2-chlorobenzene)piperazinyl]ethyl]-1,3-dimethylxanthine, a 7-[2-[4-(4-nitrobenzene)piperazinyl]ethyl]-1,3-dimethylxanthine, and a respective pharmaceutical acceptable salt thereof, wherein the inflammation is induced by a tissue necrosis factor-α in a mammal tracheal smooth muscle cell.

Preferably, the method further includes providing an additive selected from the group consisting of a pharmaceutical excipient, a diluent and a carrier.

The above aspects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 shows the respective chemical structures of KMUP-3 and KMUP-4 according to the present invention;

FIG. 2 shows the expression of inducible nitric oxide systhase, iNOS, in the tracheal smooth muscle cell (TSM) culture exposing to the TNF-α in different time period;

FIG. 3 shows the expression of sGCα1 and sGCβ1 in the tracheal smooth muscle cell (TSM) culture exposing to the TNF-α in different time period;

FIG. 4(A) shows the expression of iNOS, in the tracheal smooth muscle cell culture exposing to the TNF-α with different concentration of KMUP-1 or KMUP-3 according to the present invention;

FIG. 4(B) shows the expression of iNOS, in the tracheal smooth muscle cell culture exposing to the TNF-α with different concentration of KMUP-1, KMUP-3, zaprinast or 8-Br-cGMP;

FIG. 5 shows the expression of sGCα1 and sGCβ1 in the tracheal smooth muscle cell culture exposing to the TNF-α with KMUP-1, KMUP-3, zaprinast or 8-Br-cGMP;

FIG. 6 shows the expression of PKA and PKG in the tracheal smooth muscle cell (TSM) culture exposing to the TNF-α in different time period;

FIG. 7(A) shows the expression of PKG in the tracheal smooth muscle cell culture exposing to the TNF-α with different concentration of KMUP-1 or KMUP-3 according to the present invention;

FIG. 7(B) shows the expression of PKA in the tracheal smooth muscle cell culture exposing to the TNF-α with different concentration of KMUP-1 or KMUP-3 according to the present invention;

FIG. 8(A) shows the expression of PKG in the tracheal smooth muscle cell culture exposing to the TNF-α with KMUP-1, KMUP-3, zaprinast or 8-Br-cGMP;

FIG. 8(B) shows the expression of PKA in the tracheal smooth muscle cell culture exposing to the TNF-α with KMUP-1, KMUP-3, zaprinast or 8-Br-cGMP;

FIG. 9(A) shows the expression of COX-2 in the tracheal smooth muscle cell (TSM) culture with or without exposing to the TNF-α in different time period;

FIG. 9(B) shows the expression of COX-2 in the tracheal smooth muscle cell culture exposing to the TNF-α with a pre-treating of KMUP-1, KMUP-3, dexamethasone or NS-398;

FIG. 10(A) shows the cGMP level in the tracheal smooth muscle cell culture exposing to TNF-α with a pre-treating of KMUP-1, KMUP-3 or zaprinast;

FIG. 10(B) shows the cAMP level in the tracheal smooth muscle cell culture with or without exposing to TNF-α in a different pre-treating of isoproterenol, KMUP-1 and KMUP-3;

FIG. 11 shows the nitrite/nitrate levels in the tracheal smooth muscle cell culture exposing to the TNF-α with a pre-treating of KMUP-1, KMUP-3, zaprinast or 8-Br-cGMP;

FIG. 12 shows the PGE₂ and 6-keto-PGF_{1α} levels in the tracheal smooth muscle cell culture exposing to the TNF-α with a pre-treating of KMUP-1, KMUP-3, dexamethasone or NS-398;

FIG. 13 shows a proposed mechanism for KMPU-1 and KMPU-3 on the TNF-α-induced inflammation in the rat tracheal smooth muscle cell according to the present invention.

### DETAILED DESCRIPTION OF THE PREFERRED EMBODIMENT

The present invention will now be described more specifically with reference to the following embodiments. It is to be noted that the following descriptions of preferred embodiments of this invention are presented herein for the purposes of illustration and description only; it is not intended to be exhaustive or to be limited to the precise form disclosed.

The present invention discloses an anti-proinflammation substrate including one selected from the group consisting of a 7-[2-[4-(2-chlorobenzene)piperazinyl]ethyl]-1,3-dimethylxanthine, a 7-[2-[4-(4-nitrobenzene)piperazinyl]ethyl]-1,3-dimethylxanthine, a respective pharmaceutical acceptable salt thereof, and a combination thereof, where the mentioned xanthine-based compounds inhibit TNF-α-induced expression of iNOS in tracheal smooth muscle cells (TSMCs), involving sGC/cGMP/PKG expression pathway, but without the involvement of COX-2.

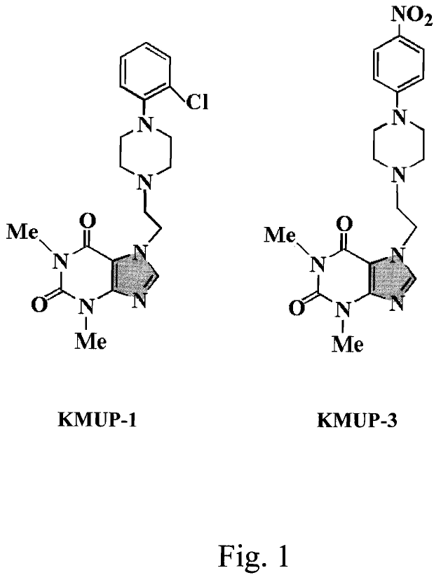

Please refer to the FIG. 1, which shows the respective chemical structures of the 7-[2-[4-(2-chlorobenzene)piperazinyl]ethyl]-1,3-dimethylxanthine (KMUP-1) and 7-[2-[4-(4-nitrobenzene)piperazinyl]ethyl]-1,3-dimethylxanthine (KMUP-3) according to the present invention.

In the following descriptions, KMUP-1 and KMUP-3 were synthesized in our laboratory. The 8-Br-cGMP, dexamethasone, indomethacin, isoproterenol, NS-398, TNF-α and zaprinast were all purchased from Sigma-Aldrich (St. Louis, Mo.). The antibodies for COX-2, iNOS and sGC were obtained from Santa Cruz Biotechnology (Santa Cruz, Calif.).

The tracheal smooth muscle cell (TSM) culture is prepared from male Wistar rats purchased from the National Laboratory Animal Breeding and Research Center (Taipei, Taiwan).

The Wistar rats were injected intraperitoneally with a lethal dose of pentobarbital. The tracheas were excised and cut longitudinally through the cartilage. Using a dissecting microscope, TSM strips were dissected from the surrounding parenchyma. The epithelium was removed from the luminal surface, and bands of TSM were gently separated from the underlying connective tissue. The TSM strips were then chopped into small sections (1 mm³) and incubated in Hank's balanced salt solution (NaCl, 138 mM; NaHCO₃, 4 mM; KCl, 5 mM; KH₂PO₄, 0.3 mM; Na₂HPO₄, 0.3 mM; glucose, 1.0 mM) with 0.05% elastase type IV and 0.2% collagenase type IV (Invitrogen, Carlsbad, Calif.) for 30 min at 37° C. with gentle shaking. The solution of dissociated smooth muscle cells was centrifuged (6 min at 500 g) and the pellet was resuspended in 1:1 Dulbecco's modified Eagle's medium-Ham's F-12 medium supplemented with 10% fetal bovine serum (FBS), 0.244% NaHCO₃, and 1% penicillin/streptomycin. Cells were cultured in a condition with or without KMUP-1, KMUP-3 and other negative or positive control agents, in 25 cm² flasks at 37° C. in humidified air containing 5% CO₂. The medium was changed every 2-3 days.

Analyses for the iNOS and sGC Expressions

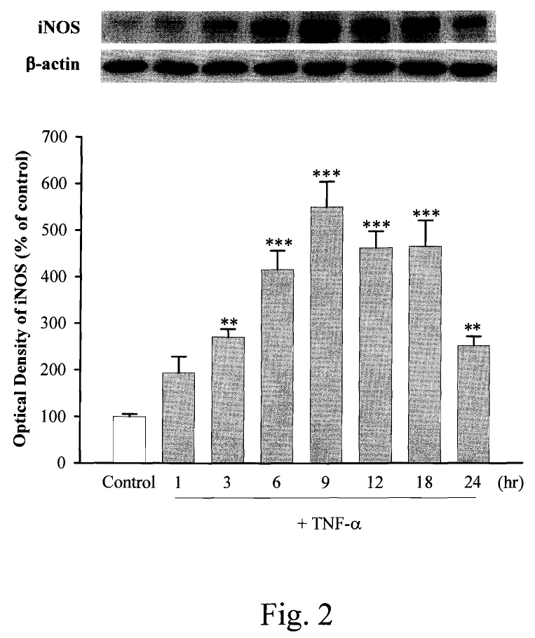

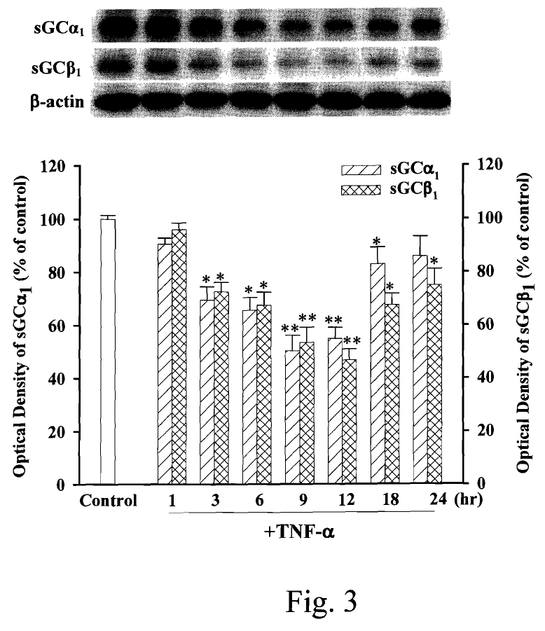

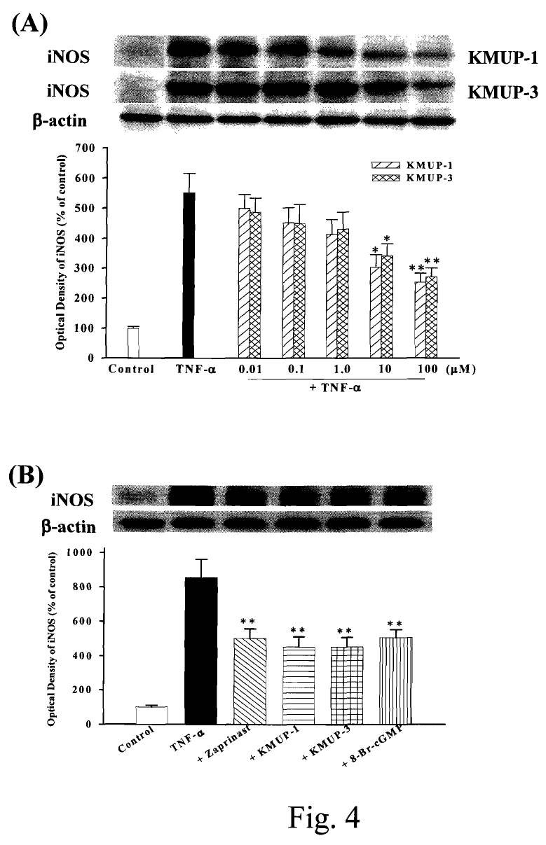

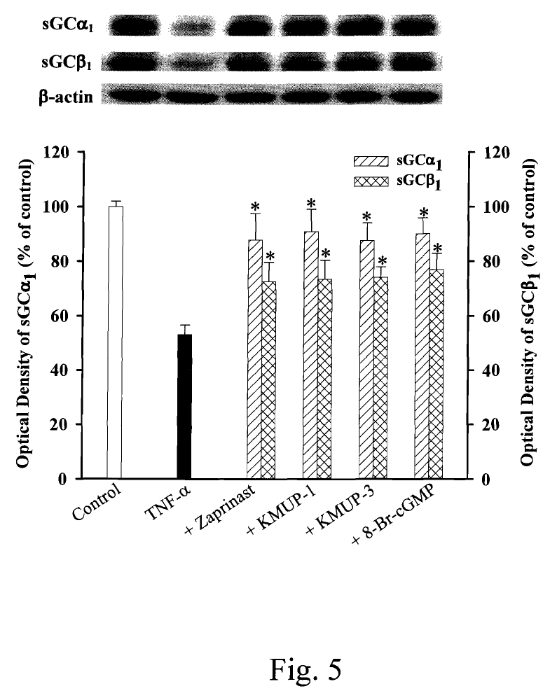

To investigate the effects of TNF-α on iNOS and sGC proteins, TSMCs were incubated with TNF-α (100 ng/ml) for 1, 3, 6, 9, 12, 18 and 24 hr, and the levels of iNOS and sGC subunit proteins were measured by immunoblotting. As shown in FIG. 2, exposure of TSMCs to TNF-α increased iNOS protein expression in a time-dependent manner, with the maximum level evident at 9 hr. In contrast to the increase expression of iNOS protein in FIG. 2, TNF-α in FIG. 3 decreased the expression of sGCα1 and sGCβ1 proteins in a time-dependent manner, with the maximal inhibition achieved at 9 hr. To investigate the effects of KMUP-1, KMUP-3, and the other PDE5 inhibitors zaprinast and exogenous 8-Br-cGMP on the TNF-α mediated pathway, TSMCs were incubated with TNF-α (100 ng/ml) and either one of the mentioned chemicals. As shown in FIG. 4(A), KMUP-1 and KMUP-3 (0.01-100 μM) inhibited TNF-α-induced increases of iNOS expression in a concentration-dependent manner. Pleaser refer to FIG. 4(B), either KMUP-1 or KMUP-3 as well as the other two PDE5 inhibitors zaprinast and exogenous 8-Br-cGMP in the same concentration (10 μM) inhibited TNF-α-induced increases of iNOS expression to a similar extent. In addition, as shown in FIG. 5, the inhibitions of TNF-α on sGCα1 and sGCβ1 proteins expressions were reversed by KMUP-1, KMUP-3, zaprinast and 8-Br-cGMP at 10 μM.

Analyses for the PKG and PKA Expressions

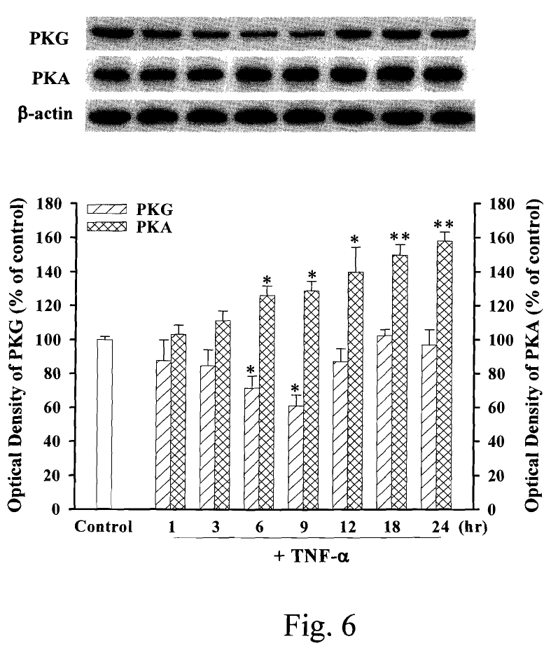

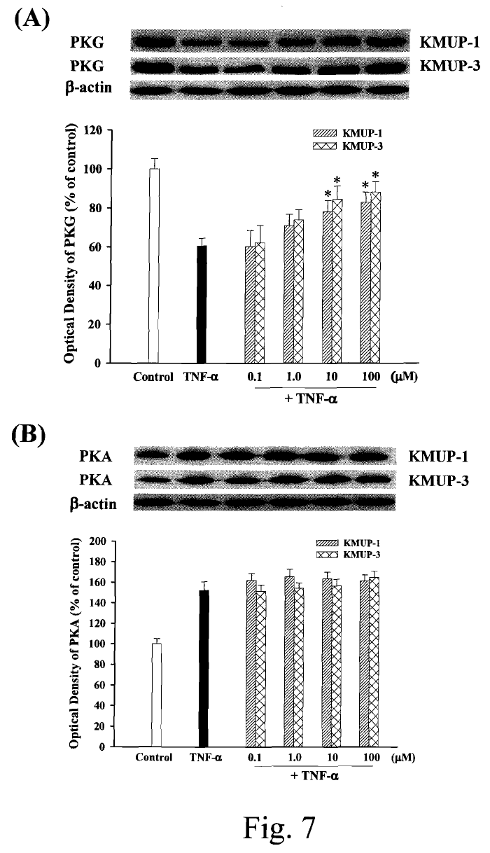

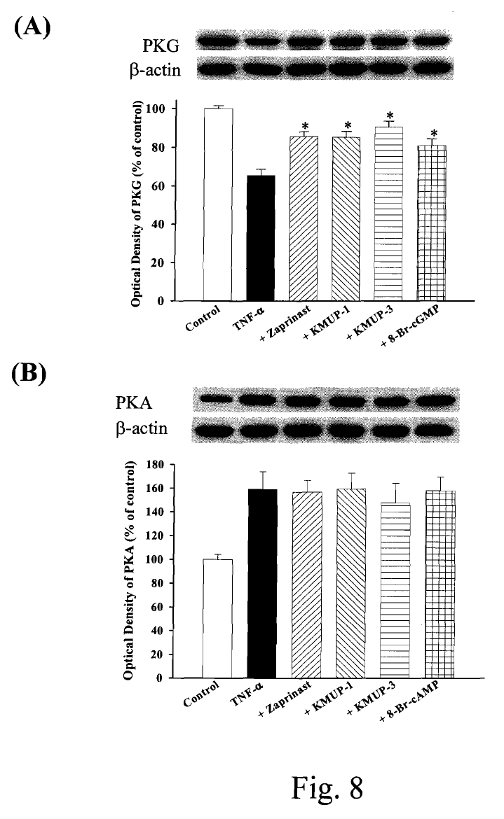

To investigate the effects of TNF-α on PKG and PKA proteins, TSMCs were incubated with TNF-α (100 ng/ml) for 1, 3, 6, 9, 12, 18 and 24 hr, and the levels of PKG and PKA proteins were measured by immunoblotting. As shown in FIG. 6, TNF-α (100 ng/ml) time-dependently increased PKA within 24 hr and significant decreased the expression of PKG protein between 6 to 9 hr. To investigate the effects of KMUP-1, KMUP-3, and the other PDE5 inhibitors zaprinast and exogenous 8-Br-cGMP on the TNF-α mediated pathway, TSMCs were incubated with TNF-α (100 ng/ml) and either one of the mentioned chemicals. As shown in FIG. 7(A), the increased expression of PKA protein by TNF-α was not further increased by KMUP-1 and KMUP-3 at a concentration of 0.1-100 μM. However, as can be seen in FIG. 7(B), the decreases of PKG protein expression by TNF-α were reversed by both KMUP-1 and KMUP-3. Please further refer to FIGS. 8(A) and 8(B), KMUP-1, KMUP-3, and the other chemicals of Zaprinast and 8-Br-cGMP at 10 μM also reversed TNF-α-induced decreases of PKG protein, whereas 8-Br-cAMP and zaprinast at 10 μM, similar to KMUP-1 and KMUP-3, could not affect TNF-α-induced increases of PKA protein.

Analyses for the COX-2 Expressions

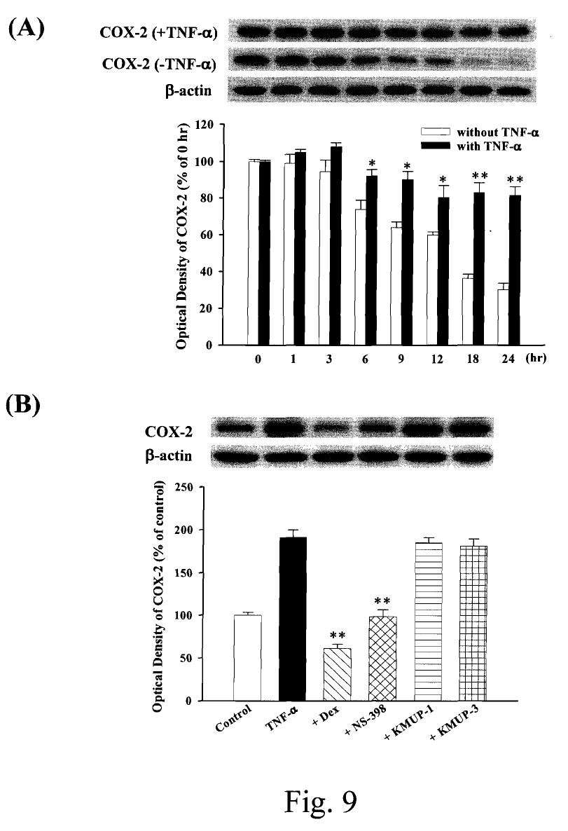

To investigate the effects of TNF-α on Cox-2 protein, TSMCs were respectively incubated with or without TNF-α (100 ng/ml) for 1, 3, 6, 9, 12, 18 and 24 hr, and the levels of COX-2 proteins were measured by immunoblotting. As shown in FIG. 9(A), in the absence of TNF-α in rat TSMCs, the expression of COX-2 showed time-dependent decreases during incubation for 24 hr. In the presence of TNF-α (100 ng/ml), the expressions of COX-2 were limited to moderate decreases after 6 hr and sustained for 24 hr in comparison with non-TNF-α challenge groups. Further, to investigate the effects of KMUP-1, KMUP-3, the other chemicals, such as the anti-inflammation agent, dexamethasone, and the COX-2 inhibitor, NS-398, on the TNF-α mediated pathway, TSMCs were incubated with TNF-α (100 ng/ml) with or without a pretreatment of either one of the mentioned chemicals. As shown in FIG. 9(B), it was clear that not KMUP-1 and KMUP-3 (10 M) but dexamethasone (1 μM) and COX-2 inhibitor NS-398 (10 μM) can significantly inhibit TNF-α-induced COX-2 expression in TSMCs.

Analyses for Cyclic Nucleotide Levels

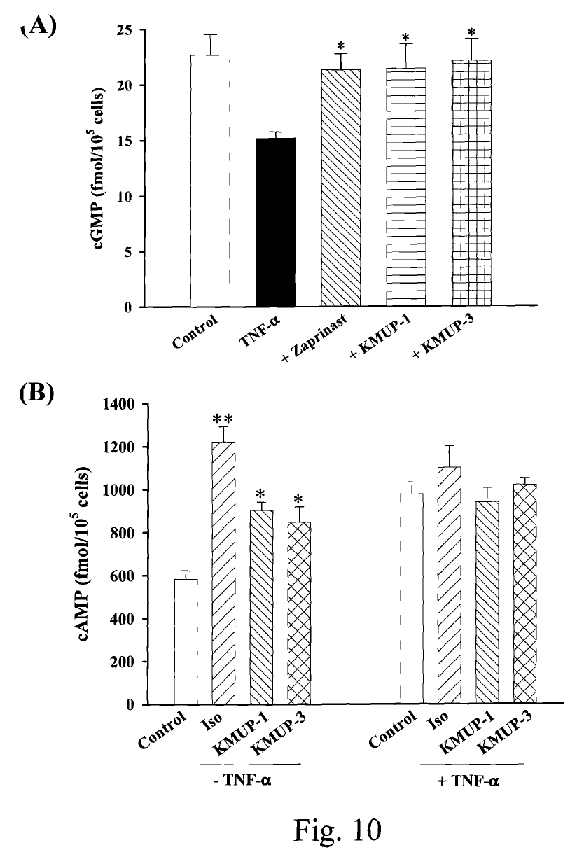

Intracellular cGMP production was decreased and reaching minimal production at 9 hr in TSMCs in the presence of TNF-α (100 ng/ml). Intracellular concentrations of cAMP and cGMP in TSMC were measured to investigate the effects of KMUP-1, KMUP-3, and the other PDE5 inhibitor zaprinast or the isoproterenol thereto. As to the level of cGMP, TSMCs were pretreated with KMUP-1, KMUP-3, and the other PDE5 inhibitor zaprinast for 20 minutes and were further incubated in the presence of TNF-α (100 ng/ml) for 9 hrs. The concentrations of cGMP of each sample were measured using cGMP-[125I] radioimmunoassay kits (GE Healthcare Bio-Sciences Corp., Piscataway, N.J.). As shown in FIG. 10(A), in the presence of KMUP-1, KMUP-3 and zaprinast (10 μM), cGMP was reversed to the basal level. As to the level of the cAMP, TSMCs were incubated with isoproterenol, KMUP-1, and KMUP-3 for 20 min, and further incubated in the absence or presence of TNF-α (100 ng/ml) for 9 hrs, where the concentrations of cAMP of each sample were measured using cAMP-[125I] radioimmunoassay kits (GE Healthcare Bio-Sciences Corp., Piscataway, N.J.). As shown in FIG. 10(B), in the absence of TNF-α, use of KMUP-1, KMUP-3 and isoproterenol (10 μM) in the culture of TSMCs significantly increased the production of cAMP, compared to the control group. However, in the presence of TNF-α, KMUP-1 and KMUP-3 could not further increase the production of cAMP, compared to the control group.

Analyses for NO Level Measured by Nitrite/Nitrate

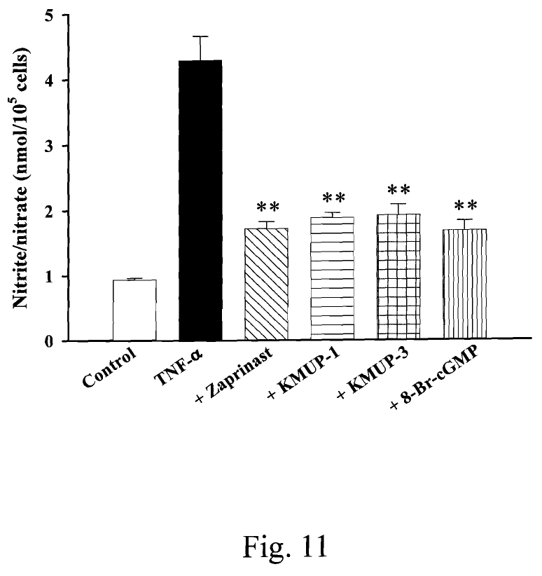

Exposure of TSMCs to TNF-α (100 ng/ml) for 9 hrs led to accumulation of nitrite and nitrate in the culture medium, indicating the release of NO. Please refer to the FIG. 11, TSMCs were pretreated with KMUP-1, KMUP-3, 8-Br-cGMP and zaprinast for 20 minutes, and were further incubated in the presence of TNF-α (100 ng/ml) for 9 hrs. The concentrations of nitrite and nitrate (stable breakdown product of NO) of each sample were analyzed using nitrite/nitrate colorimetric assay kits (Cayman Chemical Co.) and run in duplicate. As shown in FIG. 11, 8-Br-cGMP, KMUP-1, KMUP-3 and zaprinast (10 μM) all inhibited TNF-α-induced production of nitrite/nitrate, which represented the NO levels.

Analyses for PGs and COX-2 Activities

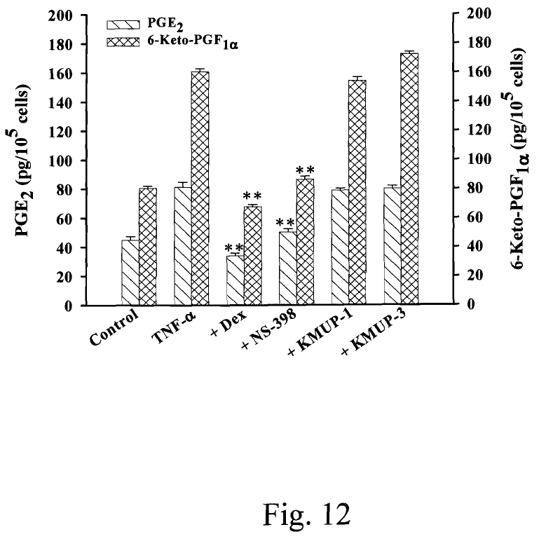

Incubation of TSMCs with TNF-α (100 ng/ml) increased the releases of PGs, PGE2 and 6-keto-PGF_{1α}, a PGI2 stable metabolite, and the greatest increases occurred were measured at 24 hr. Please refer to the FIG. 12, TSMCs were pretreated with KMUP-1 (10 μM), KMUP-3 (10 μM), dexamethasone (1 μM) and zaprinast (10 μM) for 20 minutes, and were further incubated in the presence of TNF-α (100 ng/ml) for 24 hrs, where the concentrations of PGE₂ and 6-keto-PGF_{1α} (stable metabolite of PGE2) in the culture medium were measured using PGE₂ and 6-keto-PGF_{1α} EIA assay kits (Cayman Chemical Co., Ann Arbor, Mich.) in duplicate. Dexamethasone (1 μM) and NS-398 (10 μM), a selective COX-2 inhibitor, significantly inhibited TNF-α-induced PGE₂ and 6-keto-PGF_{1α} formation. However, KMUP-1 and KMUP-3 (10 μM) did not inhibit the production of PGs, resulted from activation of COX-2.

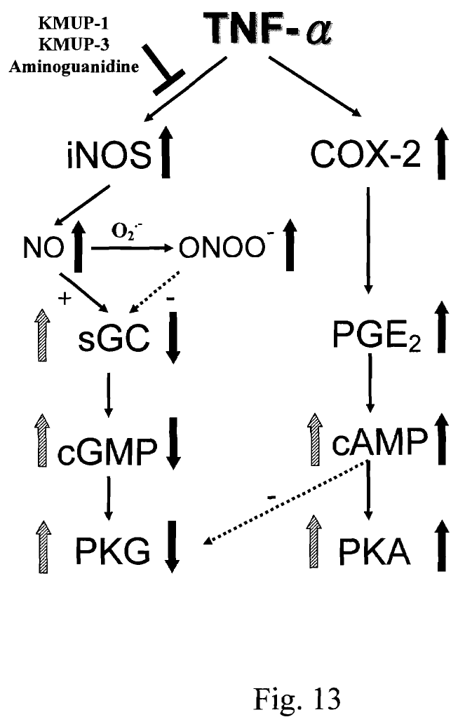

Please refer to the FIG. 13, which is a proposed mechanism for KMPU-1 and KMPU-3 on the TNF-α-induced inflammation in the rat tracheal smooth muscle cell. As shown in FIG. 13, the increased expression of iNOS by TNF-α is inhibited by KMUP-1 and KMUP-3, similar to by a iNOS inhibitor aminoguanidine; the elevated cAMP level inhibits the PKG; the elevated ONOO⁻ level inhibits the sGC; the dark thick arrow representing the protein level is increased or decreased by the TNF-α; and the damascening thick arrow representing the protein is activated or increased by either one of the KMPU-1 or KMPU-3. To sum up, either of the KMPU-1 and KMPU-3 inhibits TNF-α-induced expression of iNOS in tracheal smooth muscle cells (TSMCs), involving sGC/cGMP/PKG expression pathway, but without the involvement of COX-2.

In conclusion, under inflammatory conditions, such as in the presence of proinflammatory TNF-α in TSM, a composition including any one of the cGMP enhancing derivatives of xanthine, KMUP-1 and KMUP-3, according to the present application modulates the cross-action between PKA and PKG, by activating sGC/cGMP/PKG pathway, without the involvement of COX-2 expression. Obviously, an anti-proinflammation composition including the xanthine-based KMUP-1 or KMUP-3 with imidazole moiety reduces the cytokine-induced pro-inflammation and limited the risk of further worsening of pulmonary dysfunction.

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. An anti-inflammation substrate comprising one selected from the group consisting of a 7-[2-[4-(2-chlorobenzene)piperazinyl]ethyl]-1,3-dimethylxanthine, a 7-[2-[4-(4-nitrobenzene)piperazinyl]ethyl]-1,3-dimethylxanthine, a respective pharmaceutical acceptable salt thereof, and a combination thereof.

2. The anti-inflammation substrate as claimed in claim 1 further having one of an epithelium-derived nitric oxide enhancing activity and an endothelium-derived nitric oxide enhancing activity.

3. The anti-inflammation substrate as claimed in claim 1 further comprising one selected from the group consisting of a pharmaceutical excipient, a diluent and a carrier.

4. The anti-inflammation substrate as claimed in claim 1, used for effecting in a tracheal cGMP accumulation and relaxing a tracheal constriction by an activation of a soluble guanylate cyclase and an inhibition of the phosphodiesterase.

5. The anti-inflammation substrate as claimed in claim 1, used for preventing an airway constriction induced by a tissue necrosis factor-α by an activation of a soluable guanylate cyclase, increasing a release of cGMP and activating a protein kinase G.

6. The anti-inflammation substrate as claimed in claim 1, used for reversing a proinflammation induced by a tissue necrosis factor-α and inhibiting a lung function degeneration.

7. The anti-inflammation substrate as claimed in claim 1, used for inhibiting an inducible nitric oxide synthase (iNOS) and a protein kinase A activities and a NO production in a lung.

8. The anti-inflammation substrate as claimed in claim 1, used for preventing a soluable guanylate cyclase and a protein kinase G expression from decreasing.

9. The anti-inflammation substrate as claimed in claim 1 being a xanthine-based anti-proinflammation substrate.

10. The anti-inflammation substrate as claimed in claim 1 inhibiting the inflammation in one selected from a group consisting of a respiratory airway, a trachea and a blood vessel in a human body, wherein the inflammation comprising a pro-inflammation.

11. A method for inhibition a proinflammation induced by a tissue necrosis factor-α in a mammal tracheal smooth muscle cell, comprising administering to the mammal tracheal smooth muscle cell an inhibition-effective amount of a substrate selected from a group consisting of a 7-[2-[4-(2-chlorobenzene)piperazinyl]ethyl]-1,3-dimethylxanthine, 7-[2-[4-(4-nitrobenzene)piperazinyl]ethyl]-1,3-dimethylxanthine, a respective pharmaceutical acceptable salt thereof, and a combination thereof.

12. An anti-inflammatory use in treating one of a chronic obstructive pulmonary disease (COPD) and an asthma, comprising administering an pharmaceutically effective amount of a substrate selected from a group consisting of a 7-[2-[4-(2-chlorobenzene)piperazinyl]ethyl]-1,3-dimethylxanthine, 7-[2-[4-(4-nitrobenzene)piperazinyl]ethyl]-1,3-dimethylxanthine, a respective pharmaceutical acceptable salt thereof, and a combination thereof.

13. A method for synthesizing an anti-inflammation substrate, comprising providing a compound selected from the group consisting of a 7-[2-[4-(2-chlorobenzene)piperazinyl]ethyl]-1,3-dimethylxanthine, a 7-[2-[4-(4-nitrobenzene)piperazinyl]ethyl]-1,3-dimethylxanthine, and a respective pharmaceutical acceptable salt thereof, wherein the inflammation is induced by a tissue necrosis factor-α in a mammal tracheal smooth muscle cell.

14. The method as claimed in claim 13 further comparing providing an additive selected from the group consisting of a pharmaceutical excipient, a diluent and a carrier.
